(19) United States
(12) Patent Application Publication
(10) Pub. No.: US 2009/0264392 A1
(43) Pub. Date: Oct. 22, 2009

(54) TREATING EOSINOPHILIC ESOPHAGITIS

(75) Inventors: Roger Warndahl, Rochester, MN (US); Jeffery Alexander, Rochester, MN (US); Gianrico Farrugia, Rochester, MN (US)

Correspondence Address: WILSON, SONSINI, GOODRICH & ROSATI, 650 PAGE MILL ROAD, PALO ALTO, CA, 94304-1050, US

(73) Assignee: Meritage Pharma, Inc., San Diego, CA (US)

(21) Appl. No.: 12/426,858

(22) Filed: Apr. 20, 2009

Related U.S. Application Data
(60) Provisional application No. 61/046,691, filed on Apr. 21, 2008.

(51) Int. Cl. A61K 31/58

(52) U.S. Cl. 514/174

(57) ABSTRACT

This document provides methods and compositions suitable for treating eosinophilic esophogitis. For example, this document provides methods that involve administering a steroid and mucoadherent to a mammal (e.g., a human). Kits comprising compositions containing a steroid in combination with a mucoadherent also are provided.

### CROSS-REFERENCE

This application claims the benefit of U.S. Provisional Application No.61/046,691, filed Apr. 21, 2008, which application is incorporated herein by reference.

### BACKGROUND OF THE INVENTION

1. Technical Field

This document relates to methods and materials suitable for treating eosinophilic esophogitis and similar or related indications.

2. Background Information

Eosinophilic esophogitis (EoE) was first described by Landres in 1978, but has been increasingly recognized worldwide in recent years (Landres et al., Gastroenterology, 74:1298-1301 (1978); Liacouras, J. Pediatr. Gastroenterol. Nutr., 37 Suppl 1:S23-8 (2003); Liacouras, Clin. Gastroenterol Hepatol., 3:1198-206 (2005); Noel et al, N. Engl. J. Med., 351:940-1 (2004); and Potter et al., Gastrointest Endosc., 59:355-61 (2004)). EoE can include, in various instances, the histologic finding of greater than, e.g., 15 or 20 eosinophils per high power field on a biopsy of the esophageal mucosa (see, e.g., Arora and Yamazaki, Clin. Gastroenterol. Hepatol., 2:523-30 (2004)). The annual incidence has been estimated as high as 1:100,000 children in Ohio with an increasing prevalence reported in Switzerland over the last decade.

In children, the disease can present with various symptoms including heartburn, regurgitation, and vomiting. Adult patients primarily present with solid food dysphagia and food impaction. Various endoscopic findings have been described in EoE, and most commonly rings, longitudinal furrows, white spots, mucosal friability and strictures have been reported. Endoscopically normal appearing esophageal mucosa has been seen in up to 32 percent of children with EoE. Reports of EoE in adults suggest a small number of cases are endoscopically normal. Some have suggested the endoscopic findings of EoE are often subtle and are not easily appreciated.

### SUMMARY OF THE INVENTION

Provided in certain embodiments herein are methods and materials for treating EoE. For example, this document provides methods that include orally administering a steroid (e.g., budesonide) together with a mucoadherent to a mammal such that EoE is treated. A mucoadherent can be any compound that prolongs the contact of another molecule (e.g., a steroid) to mucous membranes, for example, to coat the surface of the esophagus. Also provided in some embodiments herein are compositions containing a steroid and a mucoadherent. Such compositions can be used as described herein to treat EoE. Also provides in certain embodiments herein is a delivery system that includes a syringe containing a composition having a steroid and a mucoadherent. The delivery system can contain tube that allows a user to deliver the composition to the back of the user's oral cavity (e.g., the back of the user's throat). Such a delivery system can allow the user to self administer a composition to the esophagus with little or no contact with the mouth, thereby preventing the development of oral thrush. The delivery systems provided herein can minimize the amount of steroid that contacts the oral pharynx.

One aspect of this document features a method for treating a mammal having eosinophilic esophogitis (EoE). The method comprising, or consists essentially of, administering a composition comprising a steroid and a mucoadherent to the mammal. The mammal can be a human. The steroid can be budesonide. The mucoadherent can be hyaluronate. The administration can be a self administration. The administration can comprise, or consist essentially of, administering the composition via a syringe device comprising a tube.

In another embodiment, provided herein is a kit for treating eosinophilic esophogitis comprising, or consisting essentially of, a syringe device and a container comprising a composition comprising a steroid and a mucoadherent. The container can house between 100 mL and 3 L of the composition. The steroid can be budesonide. The mucoadherent can be hyaluronate. The syringe device can comprise a tube.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates one embodiment of a device suitable for orally delivering a composition described herein.

### DETAILED DESCRIPTION OF THE INVENTION

Provided in certain embodiments herein are methods and materials for treating EoE. For example, in some embodiments, provided herein are methods that include orally administering a steroid (e.g., budesonide) together with a mucoadherent to a mammal such that EoE is treated. In certain embodiments, provided herein are compositions suitable for oral administration, such compositions comprising a steroid (e.g., budesonide) together with a mucoadherent to a mammal such that EoE is treated. In various embodiments, examples of steroids useful in the compositions and/or methods described herein include, by way of non-limiting example, budesonide, fluticasone, cortisone, hydrocortisone, prednisone, prednisolone, beclomethasone, methylprednisolone, dexamethasone, flunisolide, triamcinolone, mometasone, alclometasone, amcinonide, betamethasone, clobetasol, clocortolone, desonide, diflorasone, fluocinolone, fluocinonide, flurandienolide, halcinonide, halobetasol, prednicarbate, cialesonide, amcinomide, ciclesonide, clobetasone, cloprednol, cortivazol, deflazacort, deoxycorticosterone, desonide desoximetasone, diflorasone, diflucortolone, difluprednate, fluclorolone, fludrocortisone, fludroxycortide, flumetasone, fluocinolone acetonide, fluocinonide, fluocortin, fluocortolone, fluorometholone, fluperolone, fuprednidene, formocortal, halcinonide, halometasone, hydrocortisone aceponate, hydrocortisone buteprate, hydrocortisone butyrate, loteprednol, medrysone, meprednisone, methylprednisolone aceponate, mometasone furoate, paramethasone, prednicarbate, prednylidene, remexolone, tixocortol, and ulobetasol, and combinations, pharmaceutically acceptable salts and isomers thereof. In certain embodiments, examples of mucoadherents include, by way of non-limiting example, Rincinol™ (Sunstar Americas, Inc., Chicago, Ill.), sodium hyaluronate, and Gelclair bioadherent oral gel (Helsinn Healthcare S A, Lugano, Switzerland distributed by OS 1 Pharmaceuticals, Inc., Melville, N.Y.).

In some embodiments, provided herein are compositions and methods using compositions similar to Rincinol™, such compositions formulated to contain one or more of sodium hyaluronate (or another high viscosity mucopolysaccharide), hydroxyethycellulose (or a derivative of cellulose), polyvinylpyrrolidone (or similar polymers), and/or glycrrhetinic acid (e.g., an oleanolic acid from glycyrhiza). Optional ingredients include, by way of non-limiting example, aloe vera extract, propylene glycol, maltodextrin, potassium sorbate, sodium benzoate, PEG-40 hydrogenated castor oil, disodium edetate, benzalkonium chloride, flavoring, sodium saccharin, and purified water.

In some embodiments, at least one steroid (e.g., one, two, three, four, five, six, or more steroids) and at least one mucoadherent (e.g., one, two, three, four, five, six, or more mucoadherents) is formulated into a composition provided herein and/or are orally administered together to a mammal (e.g., a mouse, rat, dog, cat, horse, cow, pig, monkey, or human) to treat EoE. In certain embodiments, a composition comprising at least one steroid and at least one mucoadherent is administered to a mammal under conditions wherein the steroid has prolonged contact with the esophageal mucosa, thereby reducing at least one symptoms of EoE. In various instances, the reduction of EoE symptoms or the successful treatment of EoE is determined in any suitable manner including using a standard clinical technique including, without limitation, the resolution of endoscopic findings of rings, furrows, white spots, mucosal fragility, and strictures, a decrease in number and activity of eosinophils in esophageal biopsy specimens, and decrease dysphagia (difficulty swallowing) accessed by patient interviewing.

In some embodiments, at least one steroid and at least one mucoadherent are incorporated into a composition of the present invention. In certain embodiments, such a composition is used to treat EoE. In various embodiments, any suitable method for formulating and subsequently administering such compositions can be used. In certain instances, dosing generally is dependent and/or varies based on the severity and/or responsiveness of the EoE to steroid treatment. In certain embodiments, a method of treatment described herein can last for any suitable amount of time, e.g., from several weeks to several months. Typically, a composition described herein is administered to deliver or formulated to comprise an effective amount of steroid, e.g., between about 1 μg to about 10 mg (e.g., about 10 μg to about 5 mg, about 10 μg to about 1 mg, or about 12.2 μg to about 1 mg) of steroid per kg of body weight to a mammal (e.g., a human). In some embodiments, a composition provided herein comprises between about 0.5 μg to about 10 g (e.g., about 1 μg to about 10 g, about 5 μg to about 10 g, about 10 μg to about 10 g, about 50 μg to about 10 g, about 100 μg to about 10 g, about 200 μg to about 10 g, about 300 μg to about 10 g, about 400 μg to about 10 g, about 500 mg to about 10 g, about 750 μg to about 10 g, about 1 mg to about 10 g, about 10 mg to about 10 g, about 100 mg to about 10 g, about 500 mg to about 10 g, about 1 μg to about 1 g, about 5 μg to about 1 g, about 10 μg to about 1 g, about 50 μg to about 1 g, about 100 μg to about 1 g, about 200 μg to about 1 g, about 300 μg to about 1 g, about 400 μg to about 1 g, about 500 μg to about 1 g, about 750 μg to about 1 g, about 1 mg to about 1 g, about 5 mg to about 1 g, about 1 μg to about 100 mg, about 5 μg to about 100 mg, about 10 μg to about 100 mg, about 50 μg to about 100 mg, about 100 μg to about 100 mg, about 200 μg to about 100 mg, about 300 μg to about 100 mg, about 400 μg to about 100 mg, about 500 jag to about 100 mg, about 750 μg to about 100 mg, about 1 mg to about 100 mg, about 5 mg to about 100 mg, about 1 μg to about 10 mg, about 5 μg to about 10 mg, about 10 μg to about 10 mg, about 50 μg to about 10 mg, about 100 μg to about 10 mg, about 200 μg to about 10 mg, about 300 μg to about 10 mg, about 400 μg to about 10 mg, about 500 μg to about 10 mg, about 750 μg to about 10 mg, about 1 mg to about 10 mg, about 5 mg to about 10 mg, about 1 μg to about 1 mg, about 5 μg to about 1 mg, about 10 μg to about 1 mg, about 50 μg to about 1 mg, about 100 μg to about 1 mg, about 200 mg to about 1 mg, about 300 μg to about 1 mg, about 400 μg to about 1 mg, about 500 μg to about 1 mg, about 750 μg to about 1 mg, about 1 mg to about 1 mg, or about 5 mg to about 1 mg) of a mucoadherent (e.g., hyaluronate) per mL of the composition. In certain embodiments, a composition provided herein (e.g., those comprising at least one steroid and at least one mucoadherent) is administered once or more daily, once daily, twice daily (BID), weekly, or even less often. In an exemplary embodiment, a composition containing about 3 mg steroid (e.g., budesonide)/ 10 mL of Rincinol is administered twice a day for several weeks. In various embodiments, the total volume delivered per administration is between 1 mL and 30 mL.

In certain embodiments, provided herein is a composition comprising at least one steroid, at least one mucoadherent and, optionally, a pharmaceutically acceptable carrier. In certain instances, a “pharmaceutically acceptable carrier” includes, by way of non-limiting example, a pharmaceutically acceptable solvent, suspending agent, or any other pharmacologically inert vehicle for delivering one or more therapeutic compounds (e.g., steroids) to a subject. Pharmaceutically acceptable carriers can be liquid, and can be selected with the planned manner of administration in mind so as to provide for the desired bulk, consistency, and other pertinent transport and chemical properties, when combined with at least one of therapeutic compounds and any other components of a given pharmaceutical composition. Examples of pharmaceutically acceptable carriers include, without limitation, water; saline solution; binding agents (e.g., hydroxypropyl methylcellulose); fillers (e.g., lactose and other sugars, gelatin, or calcium sulfate); lubricants (e.g., starch, polyethylene glycol, or sodium acetate); disintegrates (e.g., starch or sodium starch glycolate); and wetting agents (e.g., sodium lauryl sulfate).

Compositions containing at least one steroid and at least one mucoadherent can be formulated into any of many possible dosage forms such as, but not limited to, liquids, liquid syrups, soft gels, liquid gels, and flowable gels. Compositions also can be formulated as suspensions in aqueous media. Aqueous suspensions further can contain substances that increase the viscosity of the suspension including, for example, sodium carboxymethylcellulose, sorbitol, and/or dextran. In certain embodiments, compositions described herein (e.g., suspensions) optionally also contain stabilizers, surfactants, suspending agents, preservatives, and flavorings.

The pharmaceutical compositions provided herein can be orally administered by self administration. For example, a human with EoE can swallow a liquid composition containing at least one steroid and at least one mucoadherent as described herein to treat EoE. In some cases, a human can fill a syringe with a pharmaceutical compositions provided herein. The tip of the syringe can be attached to piece of tubing. The human can be instructed to deliver the solution as far back into the oral cavity as possible. In some cases, the solution can be delivered directly to the back of the throat, thereby limiting steroid exposure to the oral cavity.

In some cases, a delivery system can be used to administer a composition provided herein. For example, a syringe device containing a tube or extension configured to be positioned in the back of a human's oral cavity (e.g., the back of the user's throat) can be used to deliver the composition to the esophagus with little or no contact with the mouth, thereby preventing the development of oral thrush. Examples of syringe devices include, without limitation, standard 10 cc syringes, oral syringes, and large bulb syringes. The tube can be a standard polyethylene tube having a length between 1 cm and 50 cm and a width between 1 mm and 50 mm.

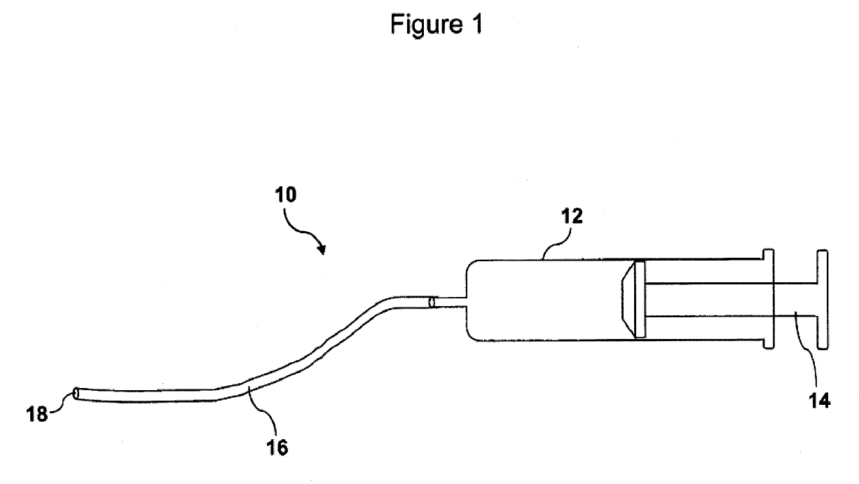

With reference to FIG. 1, syringe device 10 can contain a syringe housing 12 and a plunger 14. Plunger 14 can move within syringe housing 12 to push a fluid composition from syringe housing 12 into tube 16, which can be positioned in the back of a human's oral cavity. In some cases, tube 16 can be constructed to be a soft flexible material (e.g., polyethylene) so the user can easily position opening 18 in the back of a human's oral cavity.

The invention will be further described in the following examples, which do not limit the scope of the invention described in the claims.

### EXAMPLES

### Example 1

### Treating EoE with a Budesonide Therapy

Eighteen patients were entered into the study (mean age 41; 5 females; 13 males). 16 patients had more than 15 eos/HPF on tissue obtained from the esophagus by biopsy during endoscopy. Two patients had 10-15 eos/HPF and a very strong clinical picture to suggest EoE. Both these patients responded completely to steroids. The mean serum eosinophil count was 215. Three patients had normal endoscopies. Six patients had strictures. Concentric rings were seen in 13 patients. Longitudinal furrows were observed in one patient, and white spots were observed in another patient. Mucosal fragility was described in one patient. Six of the 18 patients exhibited erosive esophagitis. All had an Los Angeles Scale (Lundell, Gut, 45:172-180 (1999)) grade of A or B esophagitis. 14 patients underwent allergy testing. 12 patients had normal testing, while two exhibited multiple food allergies.

The following symptoms were recorded: dysphagia (18/18), heartburn (9/18), regurgitation (4/18), asthma (5/18), allergic skin disease (1/18), seasonal allergies (8/18), proton pump inhibitor (PPI) medication use to treat esophageal reflux disease (10/18), and history of food impaction (15/18).

Budesonide gel (3 mg/10 mL Rincinol) was formulated as provided below. Preparation involved accurate weighing and measuring of the ingredients. Trituration of the powder into the Rincinol liquid was performed using geometric dilution techniques. The product was stirred using a stir plate, packaged, and labeled.

The standard dose was 3 mg twice a day for one week, which was then decreased to once a day for six weeks. 11 of 18 patients received standard dosing. Six of 18 patients received an alternate treatment regiment of 3 mg twice a day for a variable treatment period of time of greater than one week. The longest of which was eight months. Ten of 18 patients exhibited a complete dysphagia response. Six of 18 exhibited a 75-99 percent symptom response, two of which were found to have strictures and eventually had total symptom resolution after dilation. One of the 18 patients exhibited a 50-74 percent response. This patient likely had concomitant a cricopharyngeal problem. His repeat endoscopy post therapy revealed a complete resolution of eosinophilia. Zero patients exhibited a 25-49 percent response, and one of 18 patients exhibited <25 percent response. This patient was found to have a tight stricture at repeat endoscopy and responded to dilation. At repeat endoscopy, her esophageal eosinophilia were resolved.

Noted side effects that did not stop the patient from administering the treatment included hoarseness (1/18) and unpleasant taste (1/18). One patient stopped treatment after experiencing myalgias.

Seven patients received fluticasone and budesonide therapy. When comparing fluticasone therapy to budesonide therapy, four of the seven patients tolerated both preparations the same. Two of the seven preferred fluticasone, and one preferred budesonide. Four of the seven patients reported that both preparations worked the same. Three of the seven reported that they responded better to budesonide after they failed to respond with fluticasone.

Four patients that were treated with budesonide have not yet reported back, or the results for one or more of these four patients are otherwise not included herein. One patient was treated who had a low eosinophil count of 4 in her esophagus and a low clinical probability of having disease. This patient, who did not respond, was excluded from the study since it is believed that she did not have EoE.

Two patients were treated for lichen planus. One patient had more than 50 percent improvement in dysphagia symptoms and had a partial endoscopic improvement. The other had complete resolution of dysphagia symptoms and a near complete endoscopic resolution.

These results demonstrate that steroids (e.g., budesonide) can be administered together with a mucoadherent to treat EoE.

### Example 2

### Therapy of Eosinophilic Esophagitis in Adults

An oral gel combining budesonide with the mucosal adherent preparation Rincinol (the combination referred to as “BRG”) was compounded. Patients with abnormal Mayo dysphagia questionnaires underwent EGD with biopsies. 16 patients with greater than 15 eos/HPF and solid food dysphagia were enrolled in the study and treated with BRG. Patients were instructed to take BRG 3mg/10 cc BID. If patients noted marked improvement at one week, they were switched to once daily BRG for 6 weeks; otherwise they were continued on BID BRG for a total of 6 weeks. Dysphagia symptoms and BRG side effects over the last two weeks of treatment were assessed by personal interview. Symptoms were evaluated on a scale of dysphagia resolution: <25%, 25-49%, 50-74%, 75-99% or complete resolution. Those patients, who had previously utilized topical fluticasone for EoE, were asked to compare BRG vs topical fluticasone with respect to treatment effect and tolerance.

See Table 1 regarding the baseline clinical features of our EoE patients. After 6 weeks of BRG therapy, all patients reported at least a 75% improvement in dysphagia symptomatology. 56% (9/16) of patients reported complete dysphagia resolution and 44% (7/16) reported a 75-99% reduction in dysphagia symptoms. Patients who transitioned from BID to once daily BRG noted no increase in dysphagia symptomatology. There was no significant difference in treatment response observed between the dosing regimens (Table 2). With respect to side effects, 13% (2/16) reported hoarseness and 6% (1/16) unpleasant taste. No oral candidiasis was observed with BRG therapy. Of the patients who had previously received fluticasone for treatment of EoE, 38% (3/8) felt BRG was more effective, 62% (5/8) had no preference and none preferred fluticasone. When asked about tolerance, 50% (4/8) of patients tolerated both preparations equally, whereas 25% (2/8) favored fluticasone and 25% (2/8) favored budesonide.

TABLE 1
Clinical Features of Patients

| | | | Allergy Testing | |
|---|---|---|---|---|
| Demographics | Symptoms | Endoscopic Findings | Results | Prior Therapies |
| Males | Food Impaction | Normal | Normal | PPI |
| 81% (13/16) | 94% (15/16) | 19% (3/16) | 71% (10/14) | 81% (13/16) |
| Females | Heartburn | Concentric Rings | Multiple Allergies | Fluticasone |
| 19% (3/16) | 44% (7/16) | 75% (12/16) | 29% (4/14) | 50% (8/16) |
| Median age 35.5 | Regurgitation | Strictures | Median Serum | |
| (Range 18-61) | 19% (3/16) | 31% (5/16) | Eosinophile Count | |
| | Asthma | Longitudinal Furrows | 0.33 (Range 0.2-0.69) | |
| | 19% (3/16) | 25% (4/16) | | |
| | Seasonal Allergies | White Spots | | |
| | 50% (8/16) | 6% (1/16) | | |

TABLE 2
Treatment Results

| | 75-99% Improvement in | 100% Improvement |
|---|---|---|
| | Dysphagia | in Dysphagia |
| Once daily BRG | 45% (5/11) | 55% (6/11) |
| BID BRG | 40% (2/5) | 60% (3/5) |

BRG effectively relieved symptoms of dysphagia in patients with esophageal eosinophilia with minimal side effects in this study. BRG may also be effective in treating EoE patients who have previously failed fluticasone. Further studies should be performed to validate these findings.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for treating a mammal having eosinophilic esophogitis, said method comprising administering a composition comprising a steroid and a mucoadherent to said mammal.

2. The method of claim 1, wherein said mammal is a human.

3. The method of claim 1, wherein said steroid is budesonide.

4. The method of claim 1, wherein said mucoadherent is hyaluronate.

5. The method of claim 1, wherein said administration is a self administration.

6. The method of claim 1, wherein said administration comprising administering said composition via a syringe device comprising a tube.

7. A kit for treating eosinophilic esophogitis comprising a syringe device and a container comprising a composition comprising a steroid and a mucoadherent.

8. The kit of claim 7, wherein said container houses between 100 mL and 3 L of said composition.

9. The kit of claim 7, wherein said steroid is budesonide.

10. The kit of claim 7, wherein said mucoadherent is hyaluronate.

11. The kit of claim 7, wherein said syringe device comprises a tube.
